SEQUENCE LISTING (4 sequences)

SEQ ID NO: 1 - DNA, 1830 bp, synthetic construct
  source 1..1830: mol_type = other DNA
atggggtccattacacccatcccactccctaagatcgatgaacccgaagagtataacaccaactacatactcttctggaaccatgtcggtttggaactcaaccgcgtaactcacactgtgggaggtcccctgacgggaccacctctctctgccagggctctgggtatgctgcacttggctattcacgacgcatacttttctatctaccctccaaccgacttcagcaccttcctctcacctaatgctgagaatgctgcgtatcgtctgcccagccctaatggtgcaaatgatgctcgccaagcagtcgctggagctgccctcaaaatgctgacttcgctctacatgaaacctgtagagacgcctaaccccaaccctggcgctaacatctccgacaatgcttacgctcagcttgccctggttatcgaccgatcagttctgaaggcgcccggtggtgttgaccgagaatcagccagcttcatgtttggtgagactgtagcagatgtcttctttgcactcctcaacgatcctcgaggtgcttcgcaggagggctaccaccctacgcccggtcgctataaatttgacgatgaacctacccatcctgtcgtcctcattccagtagaccccaacaaccccaatggccccaagaagcctttccgtcagtaccatgccccattctacggcaagaccacgaagcgttttgccacgcagagcgagcacttcctggctgacccaccgggtctgcgttccaatgcggatgagactgccgagtatgatgacgccatccgcgtcgctatcgccatgggcggtgcccaggccctcaactccaccaagcgtagcccatggcagacggcacagggtctgttctgggcctatgatgggtcaaacctcattggtacaccacctcgcttctacaaccagatcgtacgtcgcatcgcagttacgtacaagaaagaggaggaccttgcaaatagcgaagtcaacaacgcggattttgcccgcctctttgccctcgtcgacgttgcttgcaccgacgcaggtatattctcctggaaagagaagtgggagtatgaattctggcgcccactttccggtgtgcgagacgacggacgtccagaccatggagatcctttctggctcaccctcggtgccccagctactaacacaaatgacattccattcaagcctcctttcccagcttacccatctggtcacgcgacctttggcggtgccgtatttcaaatggtgcgccgatactacaacggccgcgtaggcacatggaaggacgacgagcccgacaacatcgccattgacatgatgatctcggaggaactcaacggtctgaaccgcgaccttcgccagccctacgaccccacagccccaatcgaagaccaaccaggtatcgttcgtacccgcatcgtgcgccacttcgactctgcctgggaacttatgttcgaaaacgccatttcgcgtatcttcctcggcgtccactggcgcttcgatgccgccgccgcacgcgacatccttattcccactacgaccaaggacgtctacgccgtcgacaacaacggcgcaactgtgttccagaacgtagaggatatcaggtacacgaccaagggcacgcgtgaggaccgcgagggtctctttcctatcggcggtgtgccactgggtatcgagattgcggatgagatctttaacaatggactcaagcctacacccccggagctccagcctatgccgcagcagacgcccgtgcagaagccggtaggtcagcagccggttcagggtatgtgggcggaggagcaggcgccggtgattaaggaggcgccgtaa

SEQ ID NO: 2 - protein, 609 aa, synthetic construct
MGSITPIPLPKIDEPEEYNTNYILFWNHVGLELNRVTHTVGGPLTGPPLSARALGMLHLAIHDAYFSIYPPTDFSTFLSPNAENAAYRLPSPNGANDARQAVAGAALKMLTSLYMKPVETPNPNPGANISDNAYAQLALVIDRSVLKAPGGVDRESASFMFGETVADVFFALLNDPRGASQEGYHPTPGRYKFDDEPTHPVVLIPVDPNNPNGPKKPFRQYHAPFYGKTTKRFATQSEHFLADPPGLRSNADETAEYDDAIRVAIAMGGAQALNSTKRSPWQTAQGLFWAYDGSNLIGTPPRFYNQIVRRIAVTYKKEEDLANSEVNNADFARLFALVDVACTDAGIFSWKEKWEYEFWRPLSGVRDDGRPDHGDPFWLTLGAPATNTNDIPFKPPFPAYPSGHATFGGAVFQMVRRYYNGRVGTWKDDEPDNIAIDMMISEELNGLNRDLRQPYDPTAPIEDQPGIVRTRIVRHFDSAWELMFENAISRIFLGVHWRFDAAAARDILIPTTTKDVYAVDNNGATVFQNVEDIRYTTKGTREDREGLFPIGGVPLGIEIADEIFNNGLKPTPPELQPMPQQTPVQKPVGQQPVQGMWAEEQAPVIKEAP

SEQ ID NO: 3 - protein, 609 aa, synthetic construct
MGSVTPIPLPKIDEPEEYNTNYILFWNHVGLELNRVTHTVGGPLTGPPLSARALGMLHLAIHDAYFSICPPTDFTTFLSPDTENAAYRLPSPNGANDARQAVAGAALKMLSSLYMKPVEQPNPNPGANISDNAYAQLGLVLDRSVLEAPGGVDRESASFMFGEDVADVFFALLNDPRGASQEGYHPTPGRYKFDDEPTHPVVLIPVDPNNPNGPKMPFRQYHAPFYGKTTKRFATQSEHFLADPPGLRSNADETAEYDDAVRVAIAMGGAQALNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGVNRDLRQPYDPTAPIEDQPGIVRTRIVRHFDSAWELMFENAISRIFLGVHWRFDAAAARDILIPTTTKDVYAVDNNGATVFQNVEDIRYTTRGTREDEEGLFPIGGVPLGIEIADEIFNNGLKPTPPEIQPMPQETPVQKPVGQQPVKGMWEEEQAPVVKEAP

SEQ ID NO: 4 - protein, 604 aa, synthetic construct
MGSVTPIPLPKIDEPEEYNTNYILFWNHVGLELNRVTHTVGGPLTGPPLSARALGMLHLAIHDAYFSICPPTDFTTFLSPDAENAAYRLPSPNGANDARQAVAGAALKMLSSLYMKPIEQPNPNPGANISDNAYAQLGLVLDRSVLEAPGGVDRESASFMFGEAVADVFFALLNDPRGASQEGYHPTPGRYKFDDEPTHPVVLIPVDPNNPNGPKKPFRQYHAPFYGKTTKRFATQSEHFLADPPGLRSNADETAEYDDAIRVAIAMGGAQALNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGLNRDLRQPYDPTAPIEDQPGIVRTRIVRHFDSAWELMFENAISRIFLGVHWRFDAAAARDILIPTTTKDVYAVDNNGATVFQNVEDIRYTTKGTREDREGLFPIGGVPLGIEIANEIFNNGLKPTPPEIQPMPQETPVQEPVKGMWEEEQAPIIKEAP

(19) United States
(12) Patent Application Publication
(10) Pub. No.: US 2024/0260608 A1
(43) Pub. Date: Aug. 8, 2024

(54) METHODS FOR THE REDUCTION OF METHANE PRODUCTION IN RUMINANTS

(71) Applicant: Loam Bio Pty Ltd, Orange (AU)

(72) Inventors: Abed CHAUDHURY, Orange (AU); Karen PACO, St. Paul, MN (US); Michael Anthony AYLIFFE, Canberra (AU); Thomas David LOAN, Canberra (AU); Ming LUO, Canberra (AU); Tristan YANG, St. Paul, MN (US)

(21) Appl. No.: 18/291,870

(22) PCT Filed: Aug. 2, 2022
(86) PCT No.: PCT/US2022/039095

§ 371 Date: Jan. 24, 2024

(30) Foreign Application Priority Data
Jul. 30, 2021 (AU) 2021902354

(51) Int. Cl. A23K 10/16; A23K 20/189; A23K 50/10; A61K 36/062; A61K 38/44; C12N 1/14; C12N 9/08; C12R 1/645

(52) U.S. Cl. CPC A23K 10/16; A23K 20/189; A23K 50/10; A61K 36/062; A61K 38/44; C12N 1/145; C12N 9/0065; C12Y 111/01018; C12R 2001/645

(57) ABSTRACT

The present disclosure provides methods for reducing methane emissions from a ruminant comprising administering to the ruminant a composition comprising a fungal strain, wherein the fungal strain comprises a vanadium-dependent haloperoxidase (VHPO) such as Curvularia sp. strain 4388 (NMI Accession No. V22/011149). The disclosure also provides a cell or a biologically pure culture of Curvularia sp. strain 4388 (NMI Accession No. V22/011149) or a mutant thereof having all identifying characteristics of the strain.

### CROSS REFERENCE TO RELATED APPLICATIONS

This application claims the benefit of Australian Provisional Patent Application No. 2021902354, filed on Jul. 30, 2021, the contents of which are incorporated herein by reference in their entirety.

### STATEMENT REGARDING ELECTRONIC FILING OF A SEQUENCE LISTING

A Sequence Listing in ASCII text format, submitted under 37 C.F.R. § 1.821, entitled LOAM-B001WO_Sequence_Listing.txt, 18,524 bytes in size, generated on Aug. 1, 2022, and filed via EFS-Web, is provided in lieu of a paper copy. This Sequence Listing is incorporated by reference into the specification for its disclosures.

### TECHNICAL FIELD

The present disclosure relates to the reduction of methane emissions from ruminants. Specifically, the present invention relates to methods and processes associated with the use of fungal strains that, when administered to ruminants, can reduced the methane production of methanogens in the rumen, thereby reducing methane emissions.

### BACKGROUND

Marine macroalgae, seaweeds, are producers of halogenated natural products. Biosynthesis of halogenated molecules is linked to reactive oxygen species (ROS) such as hydrogen peroxide that are substrates for haloperoxidase enzymes that promote the formation of halogenated molecules (23). It has been shown that vanadium-dependent haloperoxidases (VHPOs), halogenating enzymes that are present in seaweeds, are involved in bromoform biosynthesis. The importance of bromoform synthesis is that it can inhibit methane production (23-24). The antimethanogenic activity of bromoform was studied in vitro by isolating bioactive compound from the red seaweed Asparagopsis taxiformis.

The red macroalga Asparagopsis taxiformis has been linked to the reduction of methane (CH₄) from beef cattle by up to 99% (25). A recent study shows that A. taxiformis is a highly efficient feed supplement for CH₄ mitigation during enteric fermentation (24). The antimethanogenic compounds found in A. taxiformis are: bromoform, dibromochloromethane, bromochloroacetic acid, dibromoacetic acid and dichloromethane. However, bromoform, a halomethane, is the most abundant antimethanogenic compound found in A. taxiformis (26). On the other hand, bromoform and dibromochloromethane had the highest activity at inhibiting methane production (26).

Bromoform is a halogen that has been found to interfere with the methanogenesis pathway by serving as a competitive inhibitor or analog of methyl-coenzyme M reductase (MCR), preventing the final catalysis step (27-28).

It would be attractive, practically and commercially, to be able to reduce the methane emissions produced by ruminants, and in particular, domestic livestock, by using a source of bromoform that is easily produced with limited impact on the environment. Fungal strains expressing VHPOs present an alternative to marine macroalgae for accomplishing this objective.

### SUMMARY

To overcome limitations in the prior art described above, and to overcome other limitations that will be apparent upon reading and understanding the present specification, aspects described herein are directed towards systems, apparatuses, computer—readable media, memory, and methods for

An aspect of the present disclosure provides a method for reducing the methane emissions of a ruminant comprising administering to said ruminant a composition comprising a fungal biomass, wherein said fungal biomass comprises at least one fungal strain capable of producing bromoform.

In embodiments of the invention, the fungal strain is one that comprises one or more vanadium-dependent haloperoxidases (VHPO) genes.

In some embodiments, the disclosure provides a method for reducing methane emissions from a ruminant comprising administering to the ruminant a composition comprising a fungal strain, biomass from the fungal strain, a culture supernatant from the fungal strain, or a combination thereof, wherein the fungal strain comprises a vanadium-dependent haloperoxidase (VHPO) with an amino sequence having at least 80% identity with SEQ ID NO: 2.

In one aspect, biomass from the fungal strain is administered to the ruminant. In another aspect, a culture supernatant from the fungal strain is administered to the ruminant. In another aspect, a whole broth from the fungal strain is administered to the ruminant.

In some embodiments, the amino acid sequence of the VHPO comprises an isoleucine at position 3, a tyrosine at position 69, a serine at position 75, an asparagine at position 81, an alanine at position 82, a threonine at position 111, a threonine at position 120, an alanine at position 138, an isoleucine at position 141, a lysine at position 147, a threonine at position 163, a lysine at position 215, an isoleucine at position 261, a phenylalanine at position 288, a tyrosine at position 356, a leucine at position 447, a lysine at position 538, an arginine at position 544, a leucine at position 575, a glutamine at position 581, a glutamine at position 594, an alanine at position 598, an isoleucine at position 605, or a combination thereof.

In one embodiment, the amino acid sequence of the VHPO comprises a tyrosine at position 69, a phenylalanine at position 288, and a tyrosine at position 356. In one aspect, bromination of surface exposed aromatic residues promotes haloperoxidase activity of the VHPO.

In some aspects, the amino acid sequence of the VHPO comprises a tyrosine at position 69, a threonine at position 120, a threonine at position 163, and a glutamine at position 581.

In one aspect, the amino acid sequence of the VHPO comprises an isoleucine at position 3, a tyrosine at position 69, a serine at position 75, an asparagine at position 81, a threonine at position 111, an alanine at position 138, an isoleucine at position 141, a lysine at position 147, a phenylalanine at position 288, a tyrosine at position 356, a leucine at position 575, a glutamine at position 581, a glutamine at position 594, and an alanine at position 598.

In another aspect, the amino acid sequence of the VHPO comprises an isoleucine at position 3, a tyrosine at position 69, a serine at position 75, an asparagine at position 81, an alanine at position 82, a threonine at position 111, a threonine at position 120, an alanine at position 138, an isoleucine at position 141, a lysine at position 147, a threonine at position 163, a lysine at position 215, an isoleucine at position 261, a phenylalanine at position 288, a tyrosine at position 356, a leucine at position 447, a lysine at position 538, an arginine at position 544, a leucine at position 575, a glutamine at position 581, a glutamine at position 594, an alanine at position 598, and an isoleucine at position 605.

In other aspects, the amino acid sequence of the VHPO further comprises an arginine at position 360, a serine at position 402, a histidine at position 404, an arginine at position 490, and a histidine at position 496.

In certain embodiments, the VHPO forms a dimer and a flexible loop at the carboxy terminal region of the VHPO modulates haloperoxidase activity by modifying solvation rate and interaction with bromide (Br) species.

In other embodiments, the fungal strain belongs to the genus Curvularia.

In other aspects, the disclosure provides a method for reducing methane emissions from a ruminant comprising administering to the ruminant a composition comprising a fungal strain, biomass from the fungal strain, a culture supernatant from the fungal strain, or a combination thereof, wherein the fungal strain is Curvularia sp. strain 4388 (NMI Accession No. V22/011149) or a mutant thereof having all identifying characteristics of the strain. In some aspects, the identifying characteristics include similar VHPO activity (e.g., at least 60%, at least 70%, at least 80%, or at least 90% of that observed with Curvularia sp. strain 4388).

In some aspects, the ruminant is a member of the family Bovidae. In one aspect, the ruminant is Bos taurus.

In other embodiments, the disclosure provides a cell or a biologically pure culture of a fungal strain comprising a VHPO with an amino sequence having at least 80% identity with SEQ ID NO: 2.

In some embodiments, the disclosure provides a cell or a biologically pure culture of Curvularia sp. strain 4388 (NMI Accession No. V22/011149) or a mutant thereof having all identifying characteristics of the strain.

In other aspects, the disclosure provides an agricultural composition comprising a cell or a biologically pure culture as disclosed herein or biomass from the cell or biologically pure culture, a culture supernatant from the cell or biologically pure culture, or a combination thereof, and, optionally, an agriculturally acceptable carrier. In some aspects, the agricultural composition further comprises a cereal, starch, vegetable waste, vitamin, mineral, trace element, emulsifier, aromatizing product, binder, colorant, odorant, thickening agent, or a combination thereof.

In other aspects, the disclosure provides a use of a cell or a biologically pure culture or an agriculture composition as disclosed herein for reducing methane emissions from a ruminant.

In some aspects, the disclosure provides a method of increasing expression of VHPO in a fungal strain, the method comprising culturing the fungal strain in a culture medium comprising at least 1 g/L, 2 g/L, 3 g/L, 4 g/L, or 5 g/L glucose. In one aspect, the culture medium comprises at least 5 g/L glucose. In another aspect, the fungal strain belongs to the genus Curvularia. In another aspect, the fungal strain is Curvularia sp. strain 4388 (NMI Accession No. V22/011149) or a mutant thereof having all identifying characteristics of the strain.

Other and further aspects and features of the disclosure will be evident from reading the following detailed description of the embodiments, which are intended to illustrate, not limit, the present disclosure.

### BRIEF DESCRIPTION OF THE DRAWINGS

FIG. 1 depicts bromoform production assayed in 18 fungal strains including Curvularia sp. strain 4388 (“4388”).

FIG. 2 depicts vanadium-dependent haloperoxidase (VHPO) activity in whole broth samples from 35 fungal strains including Curvularia sp. strain 4388 (“4388”).

FIG. 3 depicts inhibition of methane production by Methanobrevibacter smithii with 0 ul, 50 ul, 100 ul, or 200 ul of Curvularia sp. strain 4388 culture supernatant with bromide (white bars) or without bromide (gray bars).

FIG. 4 depicts inhibition of methane production by Methanobrevibacter smithii by chloroform (positive control), Curvularia sp. strain 4388 culture supernatant without bromide (“Culture sup. −ve Br”; negative control), and Curvularia sp. strain 4388 culture supernatant with bromide (“Culture sup. +ve Br”) over a 40-hour time course.

FIG. 5 depicts a Clustal Omega Alignment of Curvularia sp. strain 4388 VHPO (SEQ ID NO: 2; “4388”) and Curvularia inaequalis PDB ID 1VNC VHPO (SEQ ID NO: 3; “1VNC”).

Amino acid differences are marked with black arrows.

FIG. 6 depicts a Clustal Omega Alignment of Curvularia sp. strain 4388 VHPO (SEQ ID NO: 2; “4388”), Curvularia inaequalis PDB ID 1VNC VHPO (SEQ ID NO: 3; “1VNC”), and Curvularia inaequalis strain 28 VHPO (SEQ ID NO: 4; “28”). Amino acid differences common with those indicated in FIG. 5 are marked with black arrows.

FIG. 7 depicts a three-dimensional structure of the Curvularia sp. strain 4388 VHPO protein.

FIGS. 8A, 8B, and 8C depict the three-dimensional structure of the Curvularia sp. strain 4388 VHPO protein with alpha helices shown in black, β-structures shown in black, and loops shown in black, respectively.

FIG. 9 depicts the overlap of Curvularia sp. strain 4388 VHPO and Curvularia inaequalis PDB ID 1VNC VHPO. Dark black regions represent Curvularia sp. strain 4388 VHPO, and light gray regions represent Curvularia inaequalis PDB ID 1VNC VHPO.

FIG. 10 depicts the vanadate coordination site in Curvularia sp. strain 4388 VHPO.

FIG. 11 depicts 23 amino acid variations in Curvularia sp. strain 4388 VHPO compared to Curvularia inaequalis PDB ID 1VNC VHPO (left structure) among which are three surface exposed tyrosine/phenylalanine substitutions (right structure).

FIG. 12 depicts dimerization of Curvularia sp. strain 4388 VHPO that increases stability and haloperoxidase activity.

FIG. 13 depicts the solvent accessible area of Curvularia sp. strain 4388 VHPO and energy minimized conformation. The presence of a flexible loop at the carboxy terminal modulates haloperoxidase activity.

The following description is intended only by way of example, and simply illustrates certain selected embodiments of devices, systems, and processes that are consistent with the disclosed subject matter as claimed herein.

### DETAILED DESCRIPTION

Preferred features, embodiments and variations of the invention may be discerned from the following detailed description which provides sufficient information for those skilled in the art to perform the invention. The detailed description is not to be regarded as limiting the scope of the preceding summary of the invention in any way.

In compliance with the statute, the invention has been described in language more or less specific to structural or methodical features. The term “comprises” and its variations, such as “comprising” and “comprised of” is used throughout in an inclusive sense and not to the exclusion of any additional features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Throughout the specification and claims (if present), unless the context requires otherwise, the term “substantially” or “about” will be understood to not be limited to the value for the range qualified by the terms.

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

As used herein, the term “ruminant” includes mammals that are capable of acquiring nutrients from plant-based food by fermenting it in a specialized stomach (rumen) prior to digestion, principally through microbial actions. Ruminants included cattle, goats, sheep, giraffes, yaks, deer, antelope, and others.

As used herein, the term “bovid” includes any member of the family Bovidae, which include hoofed mammals such as antelope, sheep, goats, and cattle, among others.

As used herein, the term “reducing” includes the reduction of amount of substance in comparison with a reference. For example, the reduction in the amount of total gas and/or methane produced by a ruminant animal or animals administered a composition comprising a fungal strain according to the present invention, relative to an animal or animals not administered a composition comprising a fungal strain of the present invention. The reduction can be measured in vitro with an artificial rumen system that simulates anaerobic fermentation, or in vivo with animals confined in respiration chambers. It is within the knowledge and skill of those trained in the art to assess enteric methanogenesis by a ruminant animal.

As used herein, the term “reducing methane production” refers to the reduction of methane produced in the gastro-intestinal tract. The term includes the specific volume of methane generated as a result of anaerobic fermentation, for example, in the systems described herein. Fermentation in the rumen and the gut of a ruminant gives rise to production of methane. The present invention aims to reduce this process, such as to reduce the total amount of methane produced in the gastro-intestinal tract. It is within the knowledge and skill of those trained in the art to assess methane production by a ruminant animal.

A “ruminant” is a mammal of the order Artiodactyla that digests plant-based food by initially softening and partially fermenting it within the animal's first stomach chambers, then regurgitating the semi-digested mass, now known as cud, and chewing it again. The process of rechewing the cud to further break down plant matter and stimulate digestion is called “ruminating”. Ruminants have a digestive tract with four chambers, namely the rumen, reticulum, omasum and abomasum. In the first two chambers, the rumen and the reticulum, the food is mixed with saliva and separates into layers of solid and liquid material. Solids clump together to form the cud, or bolus. The cud is then regurgitated, chewed slowly to completely mix it with saliva, which further breaks down fibers. Fiber, especially cellulose, is broken down into glucose in these chambers by symbiotic anaerobic bacteria, protozoa and fungi. The broken-down fiber, which is now in the liquid part of the contents, then passes through the rumen into the next stomach chamber, the omasum. The food in the abomasum is digested much like it would be in the monogastric stomach. Digested gut contents are finally sent to the small intestine, where the absorption of the nutrients occurs. Almost all the glucose produced by the breaking down of cellulose is used by the symbiotic bacteria. Ruminants get their energy from the volatile short chain fatty acids (VFAs) produced by the bacteria, namely acetate, propionate, butyrate, valerate, and isovalerate.

### Fungal Strains Expressing a Vanadium-Dependent Haloperoxidase

In certain aspects, the present disclosure provides a method for reducing methane emissions from a ruminant comprising administering to the ruminant a composition comprising a fungal strain, biomass from the fungal strain, a culture supernatant from the fungal strain, or a combination thereof, wherein the fungal strain comprises a vanadium-dependent haloperoxidase (VHPO).

In some aspects, the amino acid sequence of the VHPO comprises an amino sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with SEQ ID NO: 2. In one aspect, the VHPO comprises or consists of SEQ ID NO: 2.

In other aspects, the VHPO is a variant protein of the protein comprising or consisting of the amino acid sequence of SEQ ID NO: 2. Variants proteins are within the scope of the invention as long as the resulting variant protein retains similar characteristics when compared to the parent peptide. Exemplary modifications are for example conservative substitutions that will result in VHPO variants with similar characteristics to those of the parent molecules. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981). Non-conservative substitutions can be made to the VHPO variants that involve substitutions of amino acid residues between different classes of amino acids to improve properties of the VHPO variants. Whether a change in the amino acid sequence of a polypeptide or fragment thereof results in a functional homolog can be readily determined by assessing the ability of the modified polypeptide or fragment to produce a response in a fashion similar to the unmodified polypeptide or fragment using the assays described herein. Peptides, polypeptides or proteins in which more than one replacement takes place can readily be tested in the same manner.

The following conservative substitutions can be made in a VHPO from a fungal strain with the positions corresponding to those in SEQ ID NO: 2.

In certain aspects, the VHPO amino acid sequence comprises an aliphatic residue at position 3. In one aspect, the aliphatic residue at position 3 is isoleucine.

In certain aspects, the VHPO amino acid sequence comprises an aromatic residue at position 69. In one aspect, the aromatic residue at position 69 is tyrosine.

In certain aspects, the VHPO amino acid sequence comprises an aliphatic residue at position 75. In one aspect, the aliphatic residue at position 75 is serine.

In certain aspects, the VHPO amino acid sequence comprises an amide residue at position 81. In one aspect, the amide residue at position 81 is asparagine.

In certain aspects, the VHPO amino acid sequence comprises an aliphatic residue at position 82. In one aspect, the aliphatic residue at position 82 alanine.

In certain aspects, the VHPO amino acid sequence comprises an aliphatic residue at position 111. In one aspect, the aliphatic residue at position 111 is threonine.

In certain aspects, the VHPO amino acid sequence comprises an aliphatic residue at position 120. In one aspect, the aliphatic residue at position 120 is threonine.

In certain aspects, the VHPO amino acid sequence comprises an aliphatic residue at position 138. In one aspect, the aliphatic residue at position 138 is alanine.

In certain aspects, the VHPO amino acid sequence comprises an aliphatic residue at position 141. In one aspect, the aliphatic residue at position 141 is isoleucine.

In certain aspects, the VHPO amino acid sequence comprises a basic residue at position 147. In one aspect, the basic residue at position 147 is lysine.

In certain aspects, the VHPO amino acid sequence comprises an aliphatic residue at position 163. In one aspect, the aliphatic residue at position 163 is threonine.

In certain aspects, the VHPO amino acid sequence comprises a basic residue at position 215. In one aspect, the basic residue at position 215 is lysine.

In certain aspects, the VHPO amino acid sequence comprises an aliphatic residue at position 261. In one aspect, the aliphatic residue at position 261 is isoleucine.

In certain aspects, the VHPO amino acid sequence comprises an aromatic residue at position 288. In one aspect, the aromatic residue at position 288 is phenylalanine.

In certain aspects, the VHPO amino acid sequence comprises an aromatic residue at position 356. In one aspect, the aromatic residue at position 356 is tyrosine.

In certain aspects, the VHPO amino acid sequence comprises an aliphatic residue at position 447. In one aspect, the aliphatic residue at position 447 is leucine.

In certain aspects, the VHPO amino acid sequence comprises a basic residue at position 538. In one aspect, the basic residue at position 538 is lysine.

In certain aspects, the VHPO amino acid sequence comprises a basic residue at position 544. In one aspect, the basic residue at position 544 is arginine.

In certain aspects, the VHPO amino acid sequence comprises an aliphatic residue at position 575. In one aspect, the aliphatic residue at position 575 is leucine.

In certain aspects, the VHPO amino acid sequence comprises an amide residue at position 581. In one aspect, the amide residue at position 581 is glutamine.

In certain aspects, the VHPO amino acid sequence comprises an amide residue at position 594. In one aspect, the amide residue at position 594 is glutamine.

In certain aspects, the VHPO amino acid sequence comprises an aliphatic residue at position 598. In one aspect, the aliphatic residue at position 598 is alanine.

In certain aspects, the VHPO amino acid sequence comprises an aliphatic residue at position 605. In one aspect, the aliphatic residue at position 605 is isoleucine.

In certain aspects, the VHPO amino acid sequence comprises a basic residue at position 496. In one aspect, the basic residue at position 496 is histidine.

In certain aspects, the VHPO amino acid sequence comprises a basic residue at position 360. In one aspect, the basic residue at position 360 is arginine.

In certain aspects, the VHPO amino acid sequence comprises a basic residue at position 490. In one aspect, the basic residue at position 490 is arginine.

In certain aspects, the VHPO amino acid sequence comprises an aliphatic residue at position 402. In one aspect, the aliphatic residue at position 402 is serine.

In certain aspects, the VHPO amino acid sequence comprises a basic residue at position 404. In one aspect, the basic residue at position 404 is histidine.

### Animal Feed

Also disclosed herein is an animal feed comprising the compositions described herein (i.e., a composition comprising a fungal strain, biomass from the fungal strain, a culture supernatant from the fungal strain, or a combination thereof). The animal feed may be solid (e.g. powder, granules, pellets), semi-solid (e.g. gel, ointment, cream, paste) or liquid (e.g. solutions, suspensions, emulsions). The animal feed may independently be solid, semi-solid (e.g. gel, ointment, cream, paste) or liquid (e.g. solutions, suspensions, emulsions). For example, the animal feed may both be liquid or both be semi-solid or both be solid. Alternatively, the animal feed and composition may each be a different physical state. For example, the animal feed may be solid or semi-solid and the composition may be liquid. The composition may, for example, be used to “top-dress” (added on top) a ruminant feedlot ration or may be used to blend into a total mixed ration.

The composition may, for example, be added to the drinking water of the animal. In certain embodiments, the composition may be added to the drinking water of the animal immediately before ingestion, for example up to 1 hour before ingestion or up to 30 minutes before ingestion or up to 15 minutes before ingestion or up to 5 minutes before ingestion.

The three main types of animal feed include roughages, concentrates and mixed feeds. In general, roughages contain a higher percentage of crude fiber and a lower percentage of digestible nutrients than concentrates. For example, roughages may be defined as containing equal to or greater than 20 wt % crude fiber and equal to or less than 60 wt % total digestible nutrients. Roughages may include, for example, dry roughages (e.g. hay, straw, artificially dehydrated forages containing at least 90 wt % dry matter), silages (formed from green forages such as grass, alfalfa, sorghum and corn and preserved in a silo at dry matter contents of 20 to 50%), and pastures (e.g. green growing pastures providing forage that has a high water content and generally less than 30% dry matter). The two basic types of roughages include grasses and legumes. Grasses are generally higher in fiber and dry matter than legumes. Legumes are generally higher in proteins, metabolizable energy, vitamins and minerals. Concentrates contain a relatively lower percentage of crude fiber and a higher percentage of digestible nutrients than roughages. For example, concentrates may be defined as containing less than 20 wt % crude fiber and greater than 60 wt % total digestible nutrients. Concentrates may include, for example, energy-rich grains and molasses. Corn, wheat, oats, barley and milo (sorghum grain) are energy-rich grains, containing about 70 to 80 wt % total digestible nutrients.

Mixed feeds are generally a mixture of roughages and concentrates to provide “complete” balanced rations and may be either high or low in energy, protein or fiber. The disclosed compositions and fungal strains, for example, can be combined with animal feed in various amounts depending on the total amount of fungal strain, culture supernatant from the fungal strain, or combination thereof intended to be administered to the animal.

The animal feed may, for example, comprise from about 0.0001 wt % to about 10 wt % of disclosed compositions, based on the total dry weight of the animal feed. The animal feed may, for example, comprise from about 0.01 wt % to about 10 wt % of disclosed composition, based on the total dry weight of the animal feed. For example, the animal feed may comprise from about 0.001 wt % to about 9.5 wt %, or from about 0.005 wt % to about 9 wt %, or from about 0.01 wt % to about 8.5 wt %, or from about 0.05 wt % to about 8 wt %, or from about 0.1 wt % to about 7.5 wt %, or from about 0.9 wt % to about 7 wt %, or from about 1 wt % to about 6 wt %, or from about 1.5 wt % to about 5.5 wt %, or from about 2 wt % to about 5 wt %, or from about 2.5 wt % to about 4.5 wt %, or from about 3 wt % to about 4 wt % disclosed composition based on the total dry weight of the animal feed. For example, the animal feed may comprise from about 0.4 wt % to about 9.5 wt %, or from about 0.5 wt % to about 9 wt %, or from about 0.6 wt % to about 8.5 wt %, or from about 0.7 wt % to about 8 wt %, or from about 0.8 wt % to about 7.5 wt %, or from about 0.9 wt % to about 7 wt %, or from about 1 wt % to about 6 wt %, or from about 1.5 wt % to about 5.5 wt %, or from about 2 wt % to about 5 wt %, or from about 2.5 wt % to about 4.5 wt %, or from about 3 wt % to about 4 wt % disclosed composition based on the total dry weight of the animal feed.

In one embodiment, the fungal strain, biomass from the fungal strain, a culture supernatant from the fungal strain, or a combination thereof is administered at a dose of preferably at least 16.67, 10, 5, 3, 2, 1, 0.5, 0.25 0.125 or 0.067% of the organic matter administered to the ruminant animal. For example, if a 450 kg ruminant animal (e.g., steer) consumes 2.5% to 3% of its body weight per day of feed, then the fungal strain is administered at a dose proportional to the amount of organic matter administered to the ruminant. In the case of a 450 kg ruminant animal, and where 80% of the feed is organic matter, if the animal consumes about 2.5% of its body weight per day, then the fungal strain, biomass from the fungal strain, a culture supernatant from the fungal strain, or a combination thereof is administered at a dose of about 0.27, 0.18, 0.09, 0.045, 0.0225, 0.01125 or 0.00603 kg per day to result in a dose at least 3, 2, 1, 0.5, 0.25 0.125 or 0.067% of the organic matter administered to the ruminant animal.

Biological Deposit of Curvularia Sp. Strain 4388

A Biological Deposit of Curvularia sp. strain 4388 was made at the National Measurement Institute (NMI), 1/153 Bertie Street, Port Melbourne, Victoria 3207, Australia, on 10 Jun. 2022 under the provisions of the Budapest Treaty, and assigned by the International Depositary Authority the accession number V22/011149. Upon issuance of a patent, all restrictions upon the Deposit will be irrevocably removed. The Deposit is intended to meet the requirements of 37 CFR §§ 1.801-1.809. The Deposit will be maintained in the depository for a period of 30 years, or 5 years after the last request, or for the effective, enforceable life of the patent, whichever is longer, and will be replaced, if necessary, during that period. The requirements of 37 CFR §§ 1.801-1.809 are met

Any embodiment of the invention is meant to be illustrative only and is not meant to be limiting to the invention. Therefore, it should be appreciated that various other changes and modifications can be made to any embodiment described without departing from the spirit and scope of the invention.

All headings are for the convenience of the reader and should not be used to limit the meaning of the text that follows the heading, unless so specified.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Enhanced Vanadium-Dependent Haloperoxidase (VHPO) Activity in the Biomass and Supernatant Fractions from Curvularia sp. Strain 4388

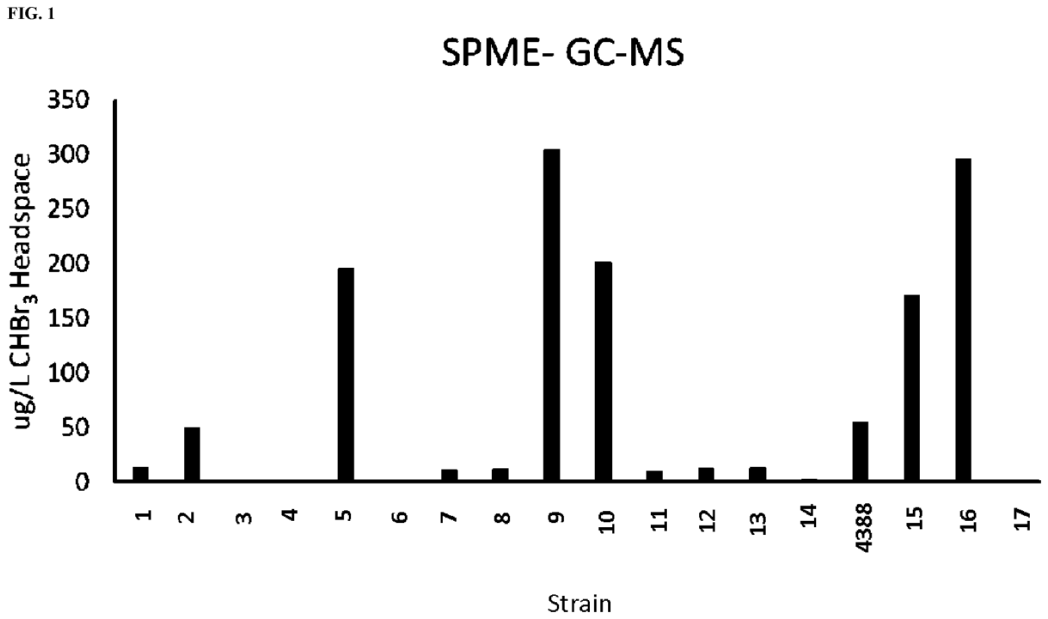

55 fungal cultures isolated from soil were tested via phenol red assay (each culture tested at 3 different day points). Fungal culture media and fungal biomass were tested separately. VHPO activity was quantified by absorbance measurements at 590 nm after both 2 hours and 24 hours initiation of the VHPO reaction with H₂O₂. The results obtained are shown in FIG. 1 for a single incubation point.

VHPO activity was detectable in most isolates with 14 isolates showing higher activity, 11 Curvularia and 3 Alternaria species. Activity was detected in both the culture media and the fungal biomass. One particular Curvularia isolate (Isolate 4388 also known as Curvularia sp. strain 4388), which showed high VHPO activity after 24 hours reaction in both the media fraction and biomass fraction, was subjected to further analysis.

For the 14 samples with high VHPO activity, including isolate 4388, more precise VHPO activity measurements were determined (i.e., enzyme kinetic assays undertaken).

Initial data collected during the optimization of the phenol red assay suggested that there was variation in VHPO rates of activity between different cultures of the same isolate. Different cultures of 4388 were pooled and used for bromoform synthesis. VHPO activity was found to be abundant in this pooled mixture after the reaction was undertaken for one week at room temperature, suggesting additional VHPO was either synthesized by the fungi or released from the biomass during this one-week bromoform synthesis reaction time.

To increase VHPO expression, the use of alternative media for isolate 4388 was investigated. The 4388 isolate was grown in triplicate cultures with different carbon sources and the VHPO activity monitored in the media. Fatty acids, glycerol, and rich complex media (PDB) did not improve or lower VHPO activity. However, increased glucose (5 g/L vs 0.5 g/L), albeit still at relatively low levels, dramatically improved both the total activity in the culture and the activity per mg of biomass. Dry mass in the increased glucose culture increased by approximately 2-fold, while VHPO activity increased by at least 20-fold.

Culture samples from isolates with VHPO activity from experiments described above were added to sealed GC-vials with KBr (20 mM), Na₃VO₄ (1 mM) Tris buffer (100 mM, pH 7.6) acetone (1 mM) and H₂O₂ (5 mM). These samples were incubated at room temperature for 1 week before analysis by GC-MS using solid-phase microextraction to monitor the presence of bromoform in the headspace. Bromoform concentration in the headspace was estimated by comparing the signals specific to bromoform with samples containing known concentrations. These results are shown in FIG. 1. Bromoform was detected in all but one of the isolates tested.

### Example 2. Increased VHPO Activity in Curvularia sp. Strain 4388 Whole Broth Samples

Fungal strains were cultured in 5 g/KL yeast extract, 1 g/L glucose, 9 uM K₂PO4, 5 uM CuSO₄, 5.5 uM FeSO₄, 8 uM MgCl₂, 5 uM ZnSO₄, 50 uM Na₃VO₄ at 28° C. at 200 rpm for 14 days. VHPO assays were conducted in 0.225 uM phenol red, 0.1 M KBr, 0.5 mM, 50 mM MOPS—pH 7.0, and initiated by the addition of 20 mM H₂O₂. Conversion of phenol red to bromophenol blue (measure of VHPO activity) was evaluated at an absorbance of 590 nm (bromophenol blue λₘₐₓ) and 430 nm (phenol red Δₘₐₓ).

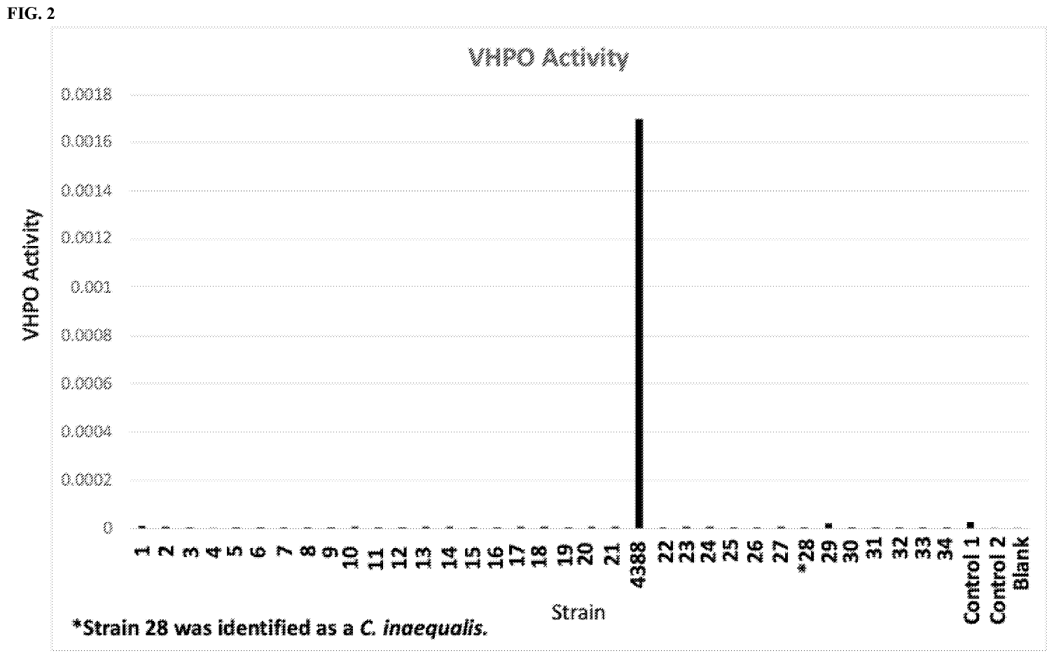

The results shown in FIG. 2 indicate that whole broth samples from Curvularia sp. strain 4388 had the greatest VHPO activity of any of the 35 fungal strains assayed, which included several Curvularia and Alternaria strains including Curvularia inaequalis strain 28.

### Example 3. Increased VHPO Activity in Curvularia sp. Strain 4388 Biomass Samples

VHPO activity from biomass samples in 55 different strains of fungi including Curvularia sp. strain 4388 were assayed. Most of the strains were Curvularia or Alternaria. For biomass measurements, total biomass from 15 mL cultures was rinsed with MilliQ water and freeze dried, and samples were homogenized with a steel ball bearing in a TissueLyser at 30 Hz for 2 min. The samples were then resuspended in 1 mL MilliQ water and vortexed. 100 uL of each sample was assayed for VHPO (200 uL final volume). Of particular interest was the high activity in 4388, matching well with the high activity previously observed in the media from this strain (see Table 1).

TABLE 1
VHPO activity assayed in biomass samples from 55 fungal
strains including Curvularia sp. strain 4388.

| Strain | VHPO Activity |
|---|---|
| 1 | −0.000002134 |
| 2 | −0.000001816 |
| 3 | −0.000001519 |
| 4 | −0.000001437 |
| 5 | −0.000001216 |
| 6 | −0.000001027 |
| 7 | −8.64255E−07 |
| 8 | −5.62677E−07 |
| 9 | −5.49111E−07 |
| 10 | −5.28156E−07 |
| 11 | −4.54917E−07 |
| 12 | −2.48047E−07 |
| 13 | −2.40734E−07 |
| 14 | −2.31356E−07 |
| 15 | −1.79104E−07 |
| 16 | −1.61124E−07 |
| 17 | −1.60013E−07 |
| 18 | −1.18752E−07 |
| 19 | −9.3623E−08 |
| 20 | −2.359E−08 |
| 21 | −2.256E−08 |
| 22 | 3.021E−08 |
| 23 | 3.5584E−08 |
| 24 | 3.6715E−08 |
| 25 | 9.7619E−08 |
| 26 | 1.67141E−07 |
| 27 | 4.47855E−07 |
| 28 | 4.70204E−07 |
| 29 | 5.79525E−07 |
| 30 | 6.15422E−07 |
| 31 | 6.26701E−07 |
| 32 | 7.00408E−07 |
| 33 | 7.53119E−07 |
| 34 | 7.79568E−07 |
| 35 | 0.000001298 |
| 36 | 0.000001302 |
| 37 | 0.000001373 |
| 38 | 0.000001498 |
| 39 | 0.000001616 |
| 40 | 0.00000173 |
| 41 | 0.000001858 |
| 42 | 0.000001869 |
| 43 | 0.000002285 |
| 44 | 0.000002577 |
| 45 | 0.000002886 |
| 46 | 0.000002985 |
| 47 | 0.000005138 |
| 48 | 0.000010828 |
| 49 | 0.000029175 |
| 50 | 0.000029502 |
| 51 | 0.0000451 |
| 52 | 0.00005977 |
| 53 | 0.0004 |
| 54 | 0.0015 |
| 4388 | 0.0022 |

### Example 4. Curvularia sp. Strain 4388 Culture Supernatant Inhibits Methanogenesis

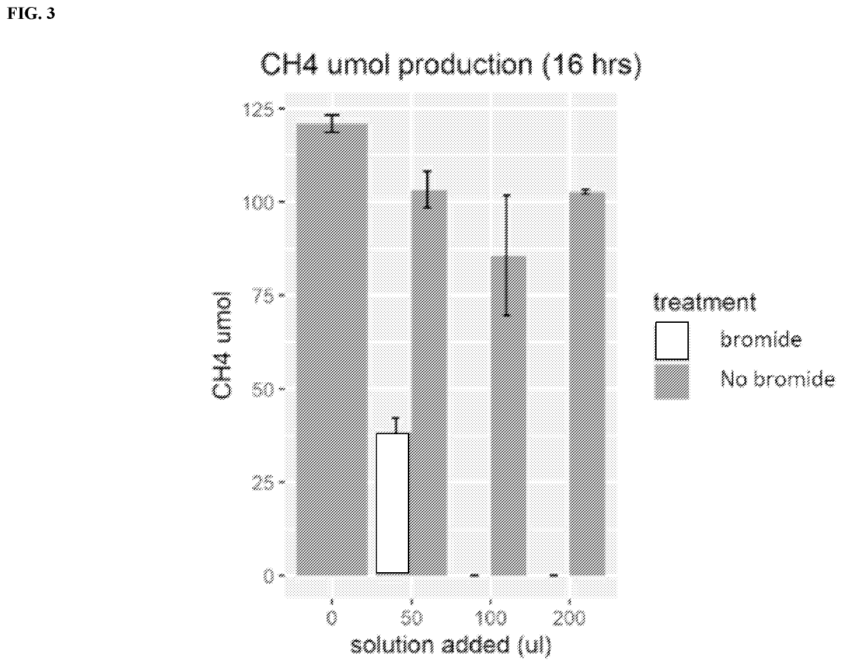

Experiments were undertaken to determine if supernatants from cultures of isolate 4388 could effectively suppress methane production by Methanobrevibacter smithii. Culture supernatants were taken from fungus grown in the presence of bromide (i.e., can potentially produce bromoform) and in the absence of bromide (i.e., cannot produce bromoform). In the first experiment, culture media was inoculated with M. smithii and culture supernatant from isolate 4388 then added. The addition of 200 ul and 100 ul of bromide culture supernatant (white bars) caused a dramatic inhibition of methane production compared with supernatants from cultures grown without bromide (gray bars) (see FIG. 3 where no significant methane production was observed with 100 ul or 200 ul of bromide culture supernatant). The lowest amount of bromide-containing culture (50 ul) also showed inhibition of methane production but less so as would be expected in a titration experiment.

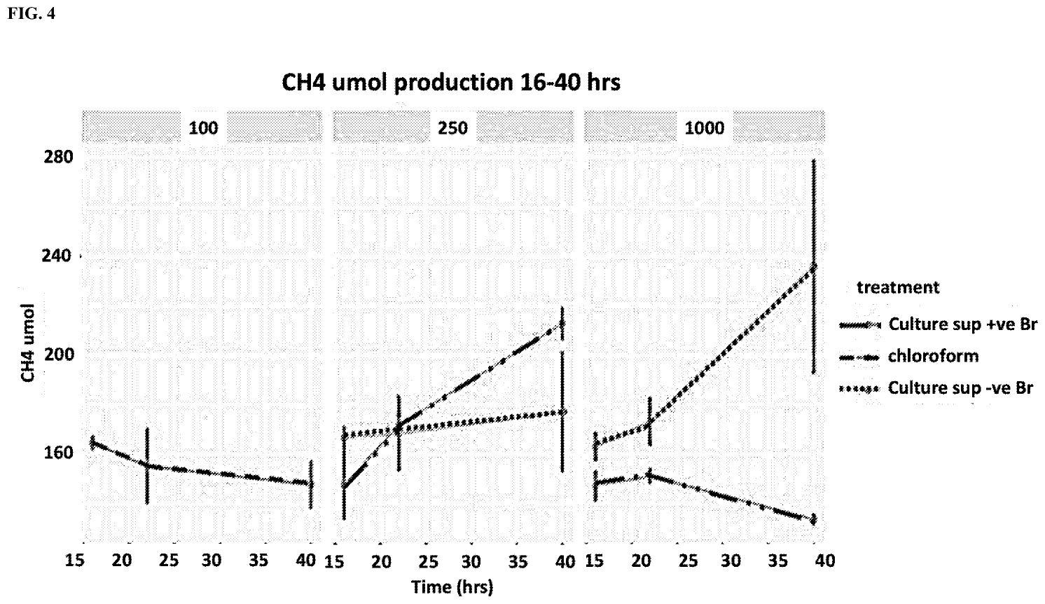

In another experiment, advanced Methanobrevibacter smithii cultures that had been grown for 16 hours were treated with 0, 250 ul, and 1000 ul of supernatant from bromide-containing (“+ve Br”) and bromide-lacking (“−ve Br”) 4388 cultures, respectively. The highest amount of bromide-containing culture (right hand graph in FIG. 4) showed a dramatic inhibition in methane production, equivalent to the addition of 100 ul of chloroform to the culture (left hand graph in FIG. 4).

Of those tested, all but two Curvularia species and three Alternaria fungal species were shown to have VHPO enzyme activity with Curvularia sp. strain 4388 showing higher activity than others. Almost all cultures tested produced bromoform with differing amounts between isolates. Culture supernatants from Curvularia sp. strain 4388 grown in the presence of bromide were shown to significantly inhibit methane production by Methanobrevibacter smithii (see FIG. 4).

### Example 5. Structural Characterization of VHPO from Curvularia sp. Strain 4388 and Methane Reduction Mechanisms

The DNA and protein sequences of VHPO from Curvularia sp. strain 4388 were determined and are shown below. To identify the VHPO sequences the genomes were assembled using the St. Petersburg genome assembler (SPAdes). RepeatMasker was used to screen the scaffolds for interspersed repeats and low complexity DNA sequences using the DFAM database (1-8).

LENGTH: 1830 bp
TYPE: DNA
FEATURE: Curvularia sp. strain 4388 VHPO
SEQ ID NO: 1
ATGGGGTCCATTACACCCATCCCACTCCCTAAGATCGATGAACCCGAAGAGTATAAC
ACCAACTACATACTCTTCTGGAACCATGTCGGTTTGGAACTCAACCGCGTAACTCAC
ACTGTGGGAGGTCCCCTGACGGGACCACCTCTCTCTGCCAGGGCTCTGGGTATGCTG
CACTTGGCTATTCACGACGCATACTTTTCTATCTACCCTCCAACCGACTTCAGCACCT
TCCTCTCACCTAATGCTGAGAATGCTGCGTATCGTCTGCCCAGCCCTAATGGTGCAA
ATGATGCTCGCCAAGCAGTCGCTGGAGCTGCCCTCAAAATGCTGACTTCGCTCTACA
TGAAACCTGTAGAGACGCCTAACCCCAACCCTGGCGCTAACATCTCCGACAATGCTT
ACGCTCAGCTTGCCCTGGTTATCGACCGATCAGTTCTGAAGGCGCCCGGTGGTGTTG
ACCGAGAATCAGCCAGCTTCATGTTTGGTGAGACTGTAGCAGATGTCTTCTTTGCAC
TCCTCAACGATCCTCGAGGTGCTTCGCAGGAGGGCTACCACCCTACGCCCGGTCGCT
ATAAATTTGACGATGAACCTACCCATCCTGTCGTCCTCATTCCAGTAGACCCCAACA
ACCCCAATGGCCCCAAGAAGCCTTTCCGTCAGTACCATGCCCCATTCTACGGCAAGA
CCACGAAGCGTTTTGCCACGCAGAGCGAGCACTTCCTGGCTGACCCACCGGGTCTGC
GTTCCAATGCGGATGAGACTGCCGAGTATGATGACGCCATCCGCGTCGCTATCGCCA
TGGGCGGTGCCCAGGCCCTCAACTCCACCAAGCGTAGCCCATGGCAGACGGCACAG
GGTCTGTTCTGGGCCTATGATGGGTCAAACCTCATTGGTACACCACCTCGCTTCTAC
AACCAGATCGTACGTCGCATCGCAGTTACGTACAAGAAAGAGGAGGACCTTGCAAA
TAGCGAAGTCAACAACGCGGATTTTGCCCGCCTCTTTGCCCTCGTCGACGTTGCTTG
CACCGACGCAGGTATATTCTCCTGGAAAGAGAAGTGGGAGTATGAATTCTGGCGCC
CACTTTCCGGTGTGCGAGACGACGGACGTCCAGACCATGGAGATCCTTTCTGGCTCA
CCCTCGGTGCCCCAGCTACTAACACAAATGACATTCCATTCAAGCCTCCTTTCCCAG
CTTACCCATCTGGTCACGCGACCTTTGGCGGTGCCGTATTTCAAATGGTGCGCCGAT
ACTACAACGGCCGCGTAGGCACATGGAAGGACGACGAGCCCGACAACATCGCCATT
GACATGATGATCTCGGAGGAACTCAACGGTCTGAACCGCGACCTTCGCCAGCCCTA
CGACCCCACAGCCCCAATCGAAGACCAACCAGGTATCGTTCGTACCCGCATCGTGC
GCCACTTCGACTCTGCCTGGGAACTTATGTTCGAAAACGCCATTTCGCGTATCTTCCT
CGGCGTCCACTGGCGCTTCGATGCCGCCGCCGCACGCGACATCCTTATTCCCACTAC
GACCAAGGACGTCTACGCCGTCGACAACAACGGCGCAACTGTGTTCCAGAACGTAG
AGGATATCAGGTACACGACCAAGGGCACGCGTGAGGACCGCGAGGGTCTCTTTCCT
ATCGGCGGTGTGCCACTGGGTATCGAGATTGCGGATGAGATCTTTAACAATGGACTC
AAGCCTACACCCCCGGAGCTCCAGCCTATGCCGCAGCAGACGCCCGTGCAGAAGCC
GGTAGGTCAGCAGCCGGTTCAGGGTATGTGGGCGGAGGAGCAGGCGCCGGTGATTA
AGGAGGCGCCGTAA
LENGTH: 609 bp
TYPE: Amino Acid
FEATURE: Curvularia sp. strain 4388 VHPO
SEQ ID NO: 2
MGSITPIPLPKIDEPEEYNTNYILFWNHVGLELNRVTHTVGGPLTGPPLSARALGMLHLAI
HDAYFSIYPPTDFSTFLSPNAENAAYRLPSPNGANDARQAVAGAALKMLTSLYMKPVET
PNPNPGANISDNAYAQLALVIDRSVLKAPGGVDRESASFMFGETVADVFFALLNDPRGA
SQEGYHPTPGRYKFDDEPTHPVVLIPVDPNNPNGPKKPFRQYHAPFYGKTTKRFATQSE
HFLADPPGLRSNADETAEYDDAIRVAIAMGGAQALNSTKRSPWQTAQGLFWAYDGSNL
IGTPPRFYNQIVRRIAVTYKKEEDLANSEVNNADFARLFALVDVACTDAGIFSWKEKWE
YEFWRPLSGVRDDGRPDHGDPFWLTLGAPATNTNDIPFKPPFPAYPSGHATFGGAVFQM
VRRYYNGRVGTWKDDEPDNIAIDMMISEELNGLNRDLRQPYDPTAPIEDQPGIVRTRIV
RHFDSAWELMFENAISRIFLGVHWRFDAAAARDILIPTTTKDVYAVDNNGATVFQNVE
DIRYTTKGTREDREGLFPIGGVPLGIEIADEIFNNGLKPTPPELQPMPQQTPVQKPVGQQP
VQGMWAEEQAPVIKEAP

The Curvularia inaequalis vanadium haloperoxidase enzymatic activity and protein structure had previously been extensively studied (17-20). Vanadium haloperoxidases contain the HVO₄²⁻ cofactor covalently attached to N² of His 496 while another five residues donate hydrogen bonds to the non-protein oxygens (21). The vanadium coordination site of VHPO Curvularia inequalis is stabilized by the amino acids: arginine 360, arginine 490, lysine 353, serine 402 and glycine 403 (21-22).

A multiple alignment of a haloperoxidase from Curvularia inaequalis and a VHPO from isolate 4388 was performed using Clustal omega (9). The primary amino acid sequence and the three-dimensional structural information for the haloperoxidase from Curvularia inaequalis was retrieved from the Protein Data Bank (PDB) (referenced herein as “Curvularia inaequalis PDB ID 1VNC VHPO”) (10). The primary amino acid sequence of another Curvularia inaequalis strain (i.e., Curvularia inaequalis strain 28) was determined as described for isolate 4388 and is also presented below.

LENGTH: 609 bp
TYPE: Amino Acid
FEATURE: Curvularia inaequalis PDB ID 1VNC VHPO
SEQ ID NO: 3
MGSVTPIPLPKIDEPEEYNTNYILFWNHVGLELNRVTHTVGGPLTGPPLSARALGMLHLA
IHDAYFSICPPTDFTTFLSPDTENAAYRLPSPNGANDARQAVAGAALKMLSSLYMKPVE
QPNPNPGANISDNAYAQLGLVLDRSVLEAPGGVDRESASFMFGEDVADVFFALLNDPR
GASQEGYHPTPGRYKFDDEPTHPVVLIPVDPNNPNGPKMPFRQYHAPFYGKTTKRFATQ
SEHFLADPPGLRSNADETAEYDDAVRVAIAMGGAQALNSTKRSPWQTAQGLYWAYDG
SNLIGTPPRFYNQIVRRIAVTYKKEEDLANSEVNNADFARLFALVDVACTDAGIFSWKE
KWEFEFWRPLSGVRDDGRPDHGDPFWLTLGAPATNTNDIPFKPPFPAYPSGHATFGGAV
FQMVRRYYNGRVGTWKDDEPDNIAIDMMISEELNGVNRDLRQPYDPTAPIEDQPGIVRT
RIVRHFDSAWELMFENAISRIFLGVHWRFDAAAARDILIPTTTKDVYAVDNNGATVFQN
VEDIRYTTRGTREDEEGLFPIGGVPLGIEIADEIFNNGLKPTPPEIQPMPQETPVQKPVGQQ
PVKGMWEEEQAPVVKEAP
LENGTH: 604 bp
TYPE: Amino Acid
FEATURE: Curvularia inaequalis strain 28 VHPO
SEQ ID NO: 4
MGSVTPIPLPKIDEPEEYNTNYILFWNHVGLELNRVTHTVGGPLTGPPLSARALGMLHLA
IHDAYFSICPPTDFTTFLSPDAENAAYRLPSPNGANDARQAVAGAALKMLSSLYMKPIEQ
PNPNPGANISDNAYAQLGLVLDRSVLEAPGGVDRESASFMFGEAVADVFFALLNDPRG
ASQEGYHPTPGRYKFDDEPTHPVVLIPVDPNNPNGPKKPFRQYHAPFYGKTTKRFATQS
EHFLADPPGLRSNADETAEYDDAIRVAIAMGGAQALNSTKRSPWQTAQGLYWAYDGS
NLIGTPPRFYNQIVRRIAVTYKKEEDLANSEVNNADFARLFALVDVACTDAGIFSWKEK
WEFEFWRPLSGVRDDGRPDHGDPFWLTLGAPATNTNDIPFKPPFPAYPSGHATFGGAVF
QMVRRYYNGRVGTWKDDEPDNIAIDMMISEELNGLNRDLRQPYDPTAPIEDQPGIVRTR
IVRHFDSAWELMFENAISRIFLGVHWRFDAAAARDILIPTTTKDVYAVDNNGATVFQNV
EDIRYTTKGTREDREGLFPIGGVPLGIEIANEIFNNGLKPTPPEIQPMPQETPVQEPVKGM
WEEEQAPIIKEAP

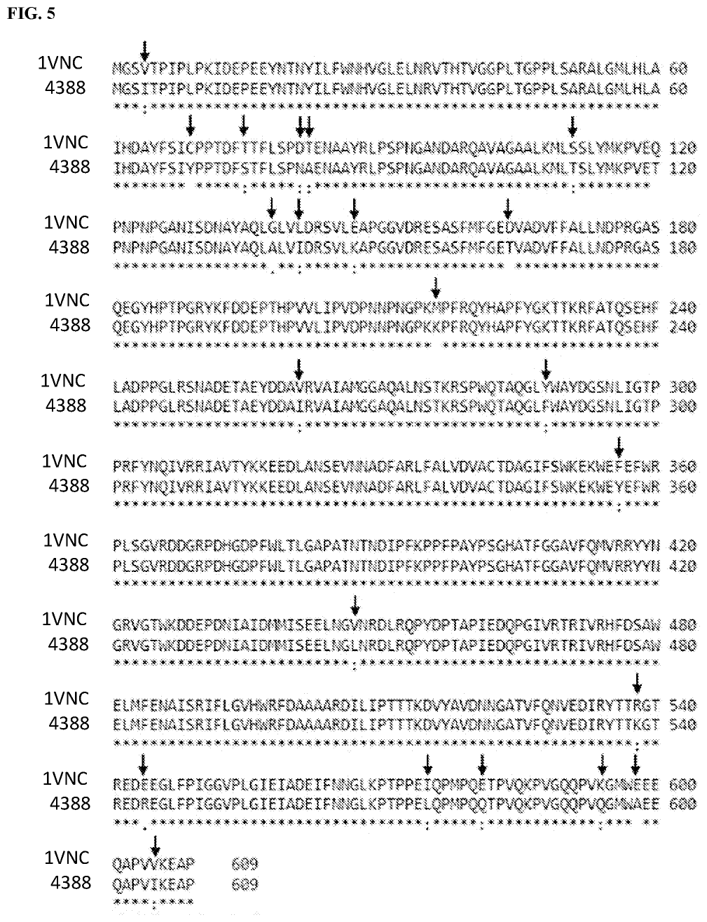

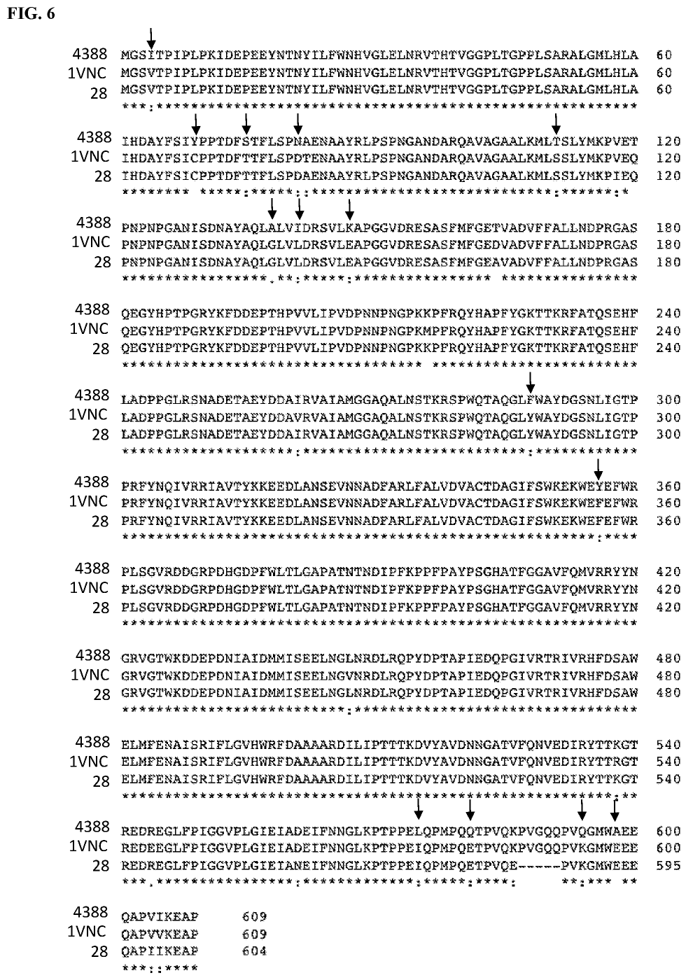

A Clustal Omega Alignment of Curvularia sp. strain 4388 VHPO and the Curvularia inaequalis PDB ID 1VNC VHPO is presented in FIG. 5. To further evaluate amino acid substitutions responsible for the increased VHPO activity in Curvularia sp. strain 4388 VHPO, a Clustal Omega Alignment of Curvularia sp. strain 4388 VHPO, Curvularia inaequalis PDB ID 1VNC VHPO, and Curvularia inaequalis strain 28 VHPO was performed (see FIG. 6).

TABLE 2
Amino acid variations between Curvularia sp. strain 4388
VHPO and Curvularia inaequalis PDB ID 1VNC VHPO. The
following substitutions were found when comparing the two
sequences. Amino acid substitutions shown in bold font were present
in Curvularia sp. strain 4388 VHPO while the corresponding
amino acid residues were conserved in both Curvularia inaequalis
PDB ID 1VNC VHPO and Curvularia inaequalis strain 28
VHPO (see FIG. 6).

| | Curvularia inaequalis PDB ID | |
|---|---|---|
| Position | 1VNC | Curvularia sp. strain 4388 |
| 3 | Val | Ile |
| 69 | Cys | Tyr |
| 75 | Thr | Ser |
| 81 | Asp | Asn |
| 82 | Thr | Ala |
| 111 | Ser | Thr |
| 120 | Gln | Thr |
| 138 | Gly | Ala |
| 141 | Leu | Ile |
| 147 | Glu | Lys |
| 163 | Asp | Thr |
| 215 | Met | Lys |
| 261 | Val | Ile |
| 288 | Tyr | Phe |
| 356 | Phe | Tyr |
| 447 | Val | Leu |
| 538 | Arg | Lys |
| 544 | Glu | Arg |
| 575 | Ile | Leu |
| 581 | Glu | Gln |
| 594 | Lys | Gln |
| 598 | Glu | Ala |
| 605 | Val | Ile |

A Clustal Omega Alignment of the amino acid sequence of Curvularia sp. strain 4388 VHPO with the amino acid sequences of VHPO from twelve representative strains selected from those evaluated in FIG. 2 including Curvularia inaequalis strain 28 was performed (data not shown). This analysis further confirmed amino acid differences responsible for the enhanced VHPO activity observed in Curvularia sp. strain 4388. The following unique amino acid variations were only present in Curvularia sp. strain 4388 VHPO and not in the VHPO from any of the twelve representative fungal strains: tyrosine at position 69, threonine at position 120, threonine at position 163, and glutamine at position 581.

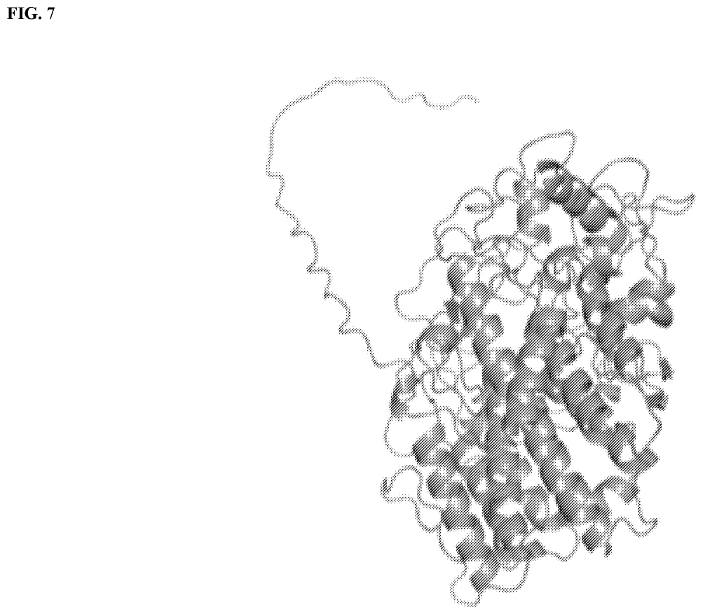

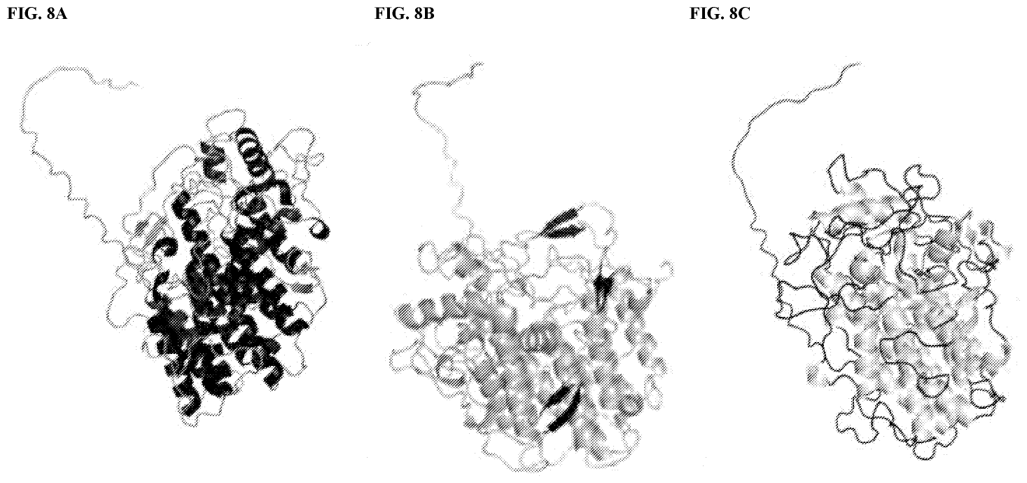

The three-dimensional structural data of Curvularia sp. strain 4388 VHPO was determined by Alphafold (11). The structure is mainly helical, a small part of β-structures, and the rest are loops. We determined disordered loops in region 120-129,180-195, 201-222, 450-463 and 567-609 by using IUPRED (12) (see FIGS. 7 and 8A-8C).

A molecular weight of 67.56 kDa and an isoelectric point of 5.11 for the Curvularia sp. strain 4388 VHPO were determined by Expasy (13).

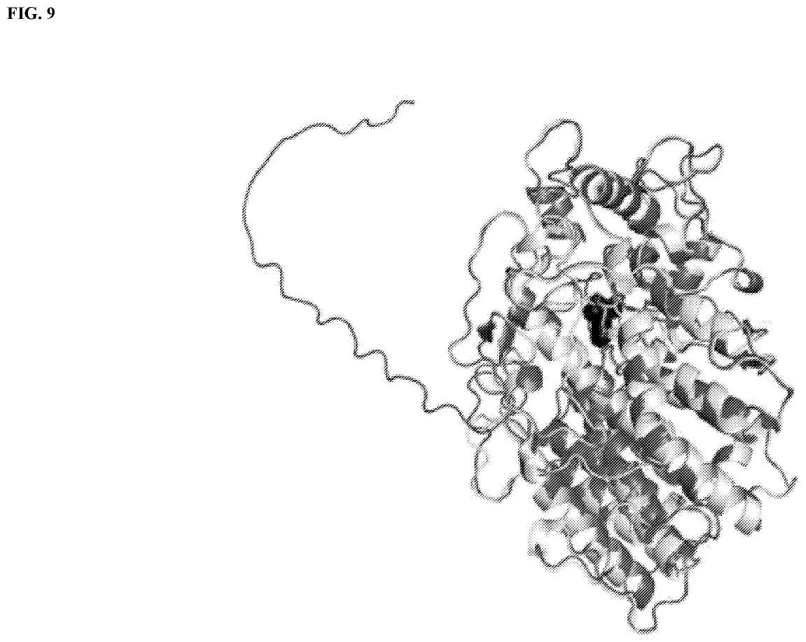

The structural similarity of Curvularia sp. strain 4388 VHPO with Curvularia inaequalis PDB ID 1VNC VHPO was determined using an alignment in pymol (14) (see FIG. 9). The root mean deviation square between the two proteins is 0.308.

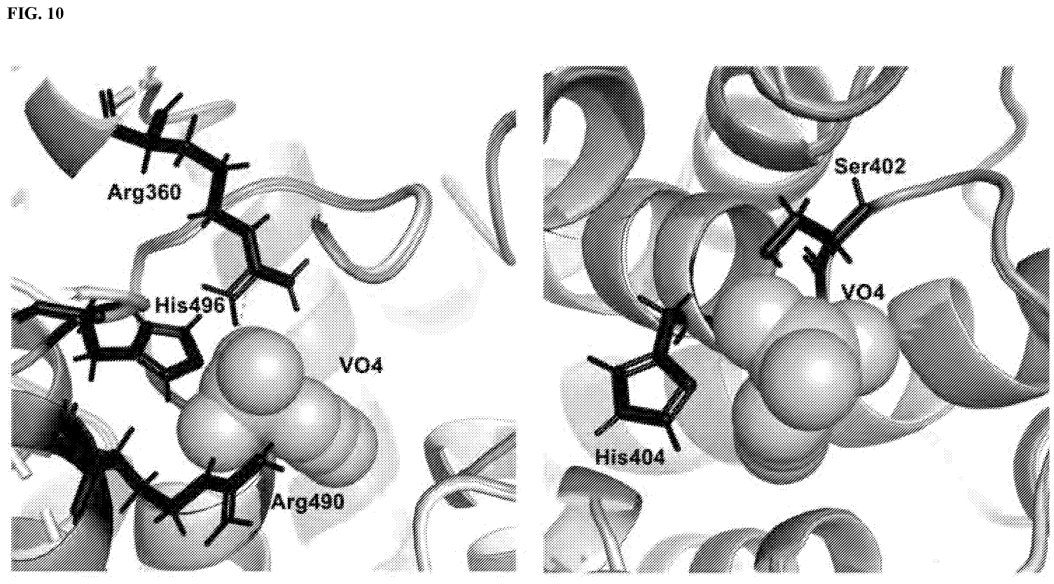

The structural conservation of the vanadate binding site in Curvularia sp. strain 4388 VHPO was determined and compared with the structure of Curvularia inaequalis PDB ID 1VNC VHPO. Histidine 496, arginine 360, arginine 490, serine 402, and histidine 404 are conserved and are amino acids that are involved in the covalent binding of vanadate with VHPO (see FIG. 10).

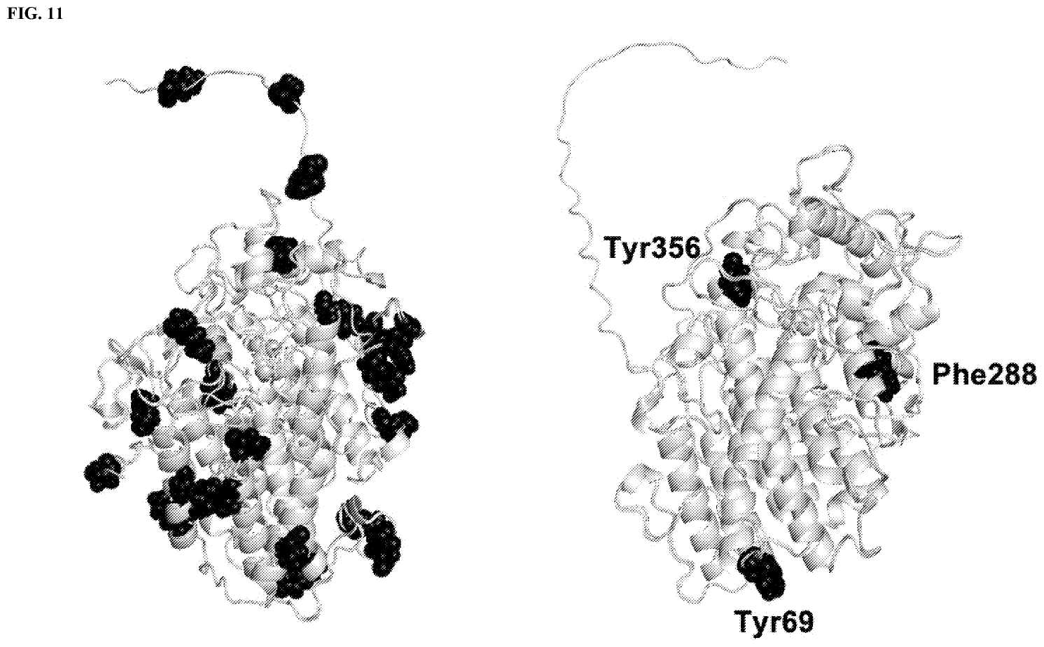

Curvularia sp. strain 4388 VHPO contains 23 amino acid variations when compared to Curvularia inaequalis PDB ID 1VNC VHPO. The location of these variations was determined in the three-dimensional structure, and it was found that the vanadate binding site is conserved. Three amino acid variations are located on the surface of the protein (i.e., tyrosine 69, phenylalanine 288, and tyrosine 356) and are aromatic residues that have been previously showed to be bound to bromide (see FIG. 11). Bromination of surface exposed aromatic residues is one of the mechanisms that promote haloperoxidase activity.

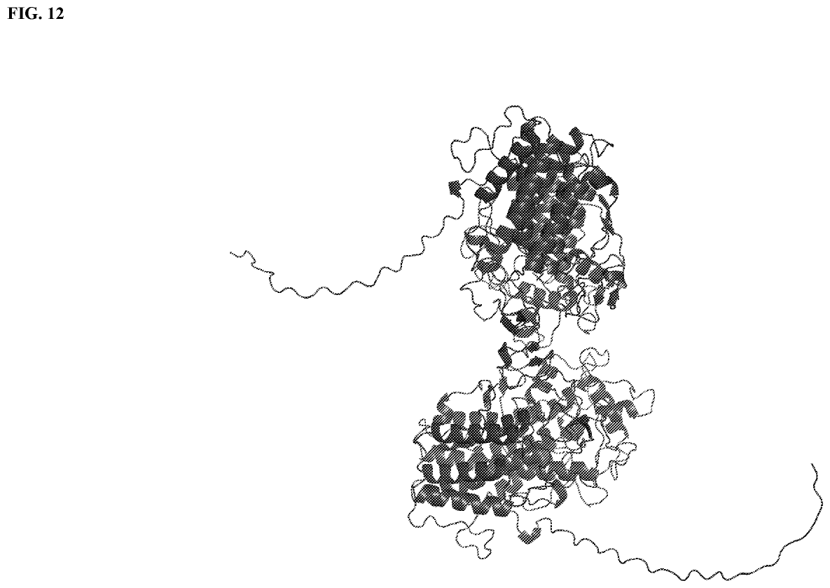

Another mechanism implicated in haloperoxidase activity is related to dimerization of monomeric VHPO subunits. The dimeric conformation of Curvularia sp. strain 4388 VHPO was determined by using Alphafold multimer (15). The three-dimensional structure of the dimeric conformation shows asymmetric binding mediated by short loop regions and short beta pleated conformations (see FIG. 12). Dimerization of Curvularia sp. strain 4388 VHPO increases stability and VHPO activity by adding two vanadate active sites in one dimeric complex.

The solvent accessible surface area (SASA) for Curvularia sp. strain 4388 VHPO was predicted and compared to Curvularia inaequalis PDB ID 1VNC VHPO.

$$Curvularia sp . strain 4388 VHPO SASA ( {nm}^{2} ) = 326.21 \\ Curvularia inaequalis PDB ID 1 VNC VHPO SASA ( {nm}^{2} ) = 245.674$$

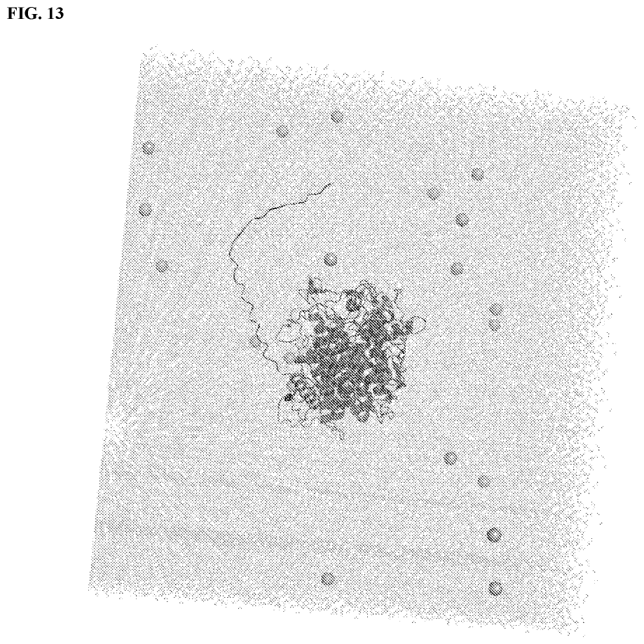

The three-dimensional structure of Curvularia sp. strain 4388 VHPO was energy minimized (steepest descent method) and solvated with water molecules and neutralized with Na⁺ and Br⁻ in an orthorhombic cell (16). The presence of a flexible loop at the carboxy terminal region modulates haloperoxidase activity by modifying solvation rate and interacting with bromide (Br⁻) species (see FIG. 13).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

1. Hubley R, Finn R D, Clements J, Eddy S R, Jones T A, Bao W, et al. The Dfam database of repetitive DNA families. Nucleic Acids Res. 2016 Jan. 4; 44 (D1):D81-9.
2. Flynn J M, Hubley R, Goubert C, Rosen J, Clark A G, Feschotte C, et al. RepeatModeler2: automated genomic discovery of transposable element families [Internet]. Genomics; 2019 November [cited 2022 Apr. 22]. Available from: http://biorxiv.org/lookup/doi/10.1101/856591). After softmasking the repeats, the Funannotate pipeline was used to predicted protein coding genes (https://funannotate.readthedocs.io/en/latest/). A number of ab initio gene predictors, including: Augustus, snap, glimmerHMM, CodingQuarry and GeneMark-ES/ET are run (Korf I. Gene finding in novel genomes. BMC Bioinformatics. 2004 May 14; 5(1):59. TigrScan and GlimmerHMM: two open source ab initio eukaryotic gene-finders| Bioinformatics| Oxford Academic [Internet]. [cited 2022 Apr. 22]. Available from: https://academic.oup.com/bioinformatics/article/20/16/2878/236797
3. Testa A C, Hane J K, Ellwood S R, Oliver R P. CodingQuarry: highly accurate hidden Markov model gene prediction in fungal genomes using RNA-seq transcripts. BMC Genomics. 2015 Mar. 11; 16(1):170.
4. Borodovsky M, Lomsadze A. Eukaryotic Gene Prediction Using GeneMark.hmm-E and GeneMark-ES. Curr Protoc Bioinforma Ed Board Andreas Baxevanis Al. 2011 September; CHAPTER:Unit.
5. Hoff K J, Stanke M. Predicting Genes in Single Genomes with AUGUSTUS. Curr Protoc Bioinforma. 2019; 65 (1):e57.
6. Brůna T, Hoff K J, Lomsadze A, Stanke M, Borodovsky M. BRAKER2: automatic eukaryotic genome annotation with GeneMark-EP+ and AUGUSTUS supported by a protein database. NAR Genomics Bioinforma [Internet]. 2021 Mar. 1 [cited 2021 Aug. 24]; 3 (1). Available from: https://doi.org/10.1093/nargab/lqaa108
7. Buchfink B, Xie C, Huson D H. Fast and sensitive protein alignment using DIAMOND. Nat Methods. 2015 January; 12(1):59-60.
8. Gotoh O. Direct mapping and alignment of protein sequences onto genomic sequence. Bioinformatics. 2008 Nov. 1; 24(21):2438-44). Finally, Evidence Modeler will take in all models to create a weighted consensus gene mode Automated eukaryotic gene structure annotation using EVidenceModeler and the Program to Assemble Spliced Alignments Genome Biology| Full Text [Internet]. [cited 2022 Apr. 22]. Available from: https://genomebiology.biomedcentral.com/articles/10.1186/gb-2008-9-1-r7).
9. Sievers et al., “Fast, Scalable Generation of High-quality Protein Multiple Sequence Alignments Using Clustal Omega.”.
10. PDB-101: Educational resources supporting molecular explorations through biology and medicine. Christine Zardecki, Shuchismita Dutta, David S. Goodsell, Robert Lowe, Maria Voigt, Stephen K. Burley. (2022) Protein Science 31: 129-140 doi:10.1002/pro.4200
11. Jumper, J et al. Highly accurate protein structure prediction with AlphaFold. Nature (2021)
12. Bálint Mészáros, Gábor Erdös, Zsuzsanna Dosztányi, IUPred2A: context-dependent prediction of protein disorder as a function of redox state and protein binding, Nucleic Acids Research, 2018; 46 (W1):W329-W337
13. (Gasteiger E., Hoogland C., Gattiker A., Duvaud S., Wilkins M. R., Appel R. D., Bairoch A.; Protein Identification and Analysis Tools on the ExPASy Server; (In) John M. Walker (ed): The Proteomics Protocols Handbook, Humana Press (2005))
14. The PyMOL Molecular Graphics System, Version 2.0 Schrödinger, LLC
15. Evans et al., “Protein Complex Prediction with AlphaFold-Multimer.”
16. Paul Bauer, Berk Hess, & Erik Lindahl. (2022). GROMACS 2022.2 Manual (2022.2). Zenodo. https://doi.org/10.5281/zenodo.6637572
17. Van Schijndel, J. W. P. M., Barnett, P., Roelse, J., Vollenbroek, E. G. M. and Wever, R. (1994), The Stability and Steady-State Kinetics of Vanadium Chloroperoxidase from the Fungus Curvularia Inaequalis. European Journal of Biochemistry, 225: 151-157. https://doi.org/10.1111/j.1432-1033.1994.00151.x
18. Dong, J. J., et al., Halofunctionalization of alkenes by vanadium chloroperoxidase from Curvularia inaequalis, Chem. Commun., 2017, 53, 6207
19. Hilda B ten Brink, Henk L Dekker, Hans E Schoemaker, Ron Wever, Oxidation reactions catalyzed by vanadium chloroperoxidase from Curvularia inaequalis, Journal of Inorganic Biochemistry, Volume 80, Issues 1-2, 2000, Pages 91-98, ISSN 0162-0134, https://doi.org/1001016/S0162-0134(00)00044-1
20. Messerschmidt, A. et al., X-ray structure of a vanadium containing enzyme: Chloroperoxidase from the fungus Curvularia inaequalis, Proc. Natl. Acad. Sci. USA, Vol. 93, pp. 392-396, January 1996 Biochemistry
21. Renirie, R., Hemrika, W., Piersma, S. R., & Wever, R. (2000). Cofactor and Substrate Binding to Vanadium Chloroperoxidase Determined by UV-VIS Spectroscopy and Evidence for High Affinity for Pervanadate †. Biochemistry, 39(5), 1133-1141. https://doi.org/10.1021/bi9221790
22. Hemrika, W., Renirie, R., Macedo-Ribeiro, S., Messerschmidt, A., & Wever, R. (1999). Heterologous Expression of the Vanadium-containing Chloroperoxidase from Curvularia inaequalis in Saccharomyces cerevisiae and Site-directed Mutagenesis of the Active Site Residues His496, Lys353, Arg360, and Arg490. Journal of Biological Chemistry, 274(34), 23820-23827. https://dio.org/10.1074/jbc.274.34.23820
23. Thapa, H. R., Lin, Z., Yi, D., Smith, J. E., Schmidt, E. W., & Agarwal, V. (2020). Genetic and Biochemical Reconstitution of Bromoform Biosynthesis in Asparagopsis Lends Insights into Seaweed Reactive Oxygen Species Enzymology. ACS Chemical Biology, 15(6), 1662-1670. https://dio.org/10.1021/schembio.0e00299
24. Machado, L., Magnusson, M., Paul, N. A., de Nys, R., & Tomkins, N. (2014). Effects of Marine and Freshwater Macroalgae on In Vitro Total Gas and Methane Production. PLOS ONE, 9(1), e85289. https://dio.org/10.1371/journal.pone.0085289
25. Roque, B. M., Brooke, C. G., Ladau, J., Polley, T., Marsh, L. J., Najafi, N., Pandey, P., Singh, L., Kinley, R., Salwen, J. K., Eloe-Fadrosh, E., Kebreab, E., & Hess, M. (2019). Effect of the macroalgae Asparagopsis taxiformis on methane production and rumen microbiome assemblage. Animal Microbiome, 1(1), 3. https://doi.org/10.1186/s42523-019-0004-4
26. Machado, L., Magnusson, M., Paul, N. A., Kinley, R., de Nys, R., & Tomkins, N. (2016). Identification of bioactives from the red seaweed Asparagopsis taxiformis that promote antimethanogenic activity in vitro. Journal of Applied Phycology, 28(5), 3117-3126. https://doi.org/10.1007/s10811-016-0830-7
27. Goel, G., Makkar, H. P. S., & Becker, K. (2009). Inhibition of methanogens by bromochloromethane: Effects on microbial communities and rumen fermentation using batch and continuous fermentations. British Journal of Nutrition, 101(10), 1484. https://dio.org/10.1017/S0007114508076198
28. Honan, M., Feng, X., Tricarico, J. M., & Kebreab, E. (2021). Feed additives as a strategic approach to reduce enteric methane production in cattle: Modes of action, effectiveness and safety. Animal Production Science. https://doi.org/10.1071/AN20295

1. A method for reducing methane emissions from a ruminant comprising administering to the ruminant a composition comprising a fungal strain, biomass from the fungal strain, a culture supernatant from the fungal strain, or a combination thereof,
wherein the fungal strain comprises a vanadium-dependent haloperoxidase (VHPO) with an amino sequence having at least 80% identity with SEQ ID NO: 2.

2. The method of claim 1, wherein the amino acid sequence of the VHPO comprises an isoleucine at position 3, a tyrosine at position 69, a serine at position 75, an asparagine at position 81, an alanine at position 82, a threonine at position 111, a threonine at position 120, an alanine at position 138, an isoleucine at position 141, a lysine at position 147, a threonine at position 163, a lysine at position 215, an isoleucine at position 261, a phenylalanine at position 288, a tyrosine at position 356, a leucine at position 447, a lysine at position 538, an arginine at position 544, a leucine at position 575, a glutamine at position 581, a glutamine at position 594, an alanine at position 598, an isoleucine at position 605, or a combination thereof.

3. The method of claim 2, wherein the amino acid sequence of the VHPO comprises a tyrosine at position 69, a phenylalanine at position 288, and a tyrosine at position 356.

4. The method of claim 3, wherein bromination of surface exposed aromatic residues promotes haloperoxidase activity of the VHPO.

5. The method of claim 2, wherein the amino acid sequence of the VHPO comprises a tyrosine at position 69, a threonine at position 120, a threonine at position 163, and a glutamine at position 581.

6. The method of claim 2, wherein the amino acid sequence of the VHPO comprises an isoleucine at position 3, a tyrosine at position 69, a serine at position 75, an asparagine at position 81, a threonine at position 111, an alanine at position 138, an isoleucine at position 141, a lysine at position 147, a phenylalanine at position 288, a tyrosine at position 356, a leucine at position 575, a glutamine at position 581, a glutamine at position 594, and an alanine at position 598.

7. The method of claim 2, wherein the amino acid sequence of the VHPO comprises an isoleucine at position 3, a tyrosine at position 69, a serine at position 75, an asparagine at position 81, an alanine at position 82, a threonine at position 111, a threonine at position 120, an alanine at position 138, an isoleucine at position 141, a lysine at position 147, a threonine at position 163, a lysine at position 215, an isoleucine at position 261, a phenylalanine at position 288, a tyrosine at position 356, a leucine at position 447, a lysine at position 538, an arginine at position 544, a leucine at position 575, a glutamine at position 581, a glutamine at position 594, an alanine at position 598, and an isoleucine at position 605.

8. The method of claim 1, wherein the amino acid sequence of the VHPO further comprises an arginine at position 360, a serine at position 402, a histidine at position 404, an arginine at position 490, and a histidine at position 496.

9. (canceled)

10. The method of claim 1, wherein the fungal strain belongs to the genus Curvularia.

11. (canceled)

12. The method of claim 1, wherein the ruminant is a member of the family Bovidae.

13. The method of claim 12, wherein the ruminant is Bos taurus.

14. A cell or a biologically pure culture of a fungal strain comprising a VHPO with an amino sequence having at least 80% identity with SEQ ID NO: 2.

15. The cell or biologically pure culture of claim 14, wherein the amino acid sequence of the VHPO comprises an isoleucine at position 3, a tyrosine at position 69, a serine at position 75, an asparagine at position 81, an alanine at position 82, a threonine at position 111, a threonine at position 120, an alanine at position 138, an isoleucine at position 141, a lysine at position 147, a threonine at position 163, a lysine at position 215, an isoleucine at position 261, a phenylalanine at position 288, a tyrosine at position 356, a leucine at position 447, a lysine at position 538, an arginine at position 544, a leucine at position 575, a glutamine at position 581, a glutamine at position 594, an alanine at position 598, an isoleucine at position 605, or a combination thereof.

16. The cell or biologically pure culture of claim 15, wherein the amino acid sequence of the VHPO comprises a tyrosine at position 69, a phenylalanine at position 288, and a tyrosine at position 356.

17. The cell or biologically pure culture of claim 15, wherein the amino acid sequence of the VHPO comprises a tyrosine at position 69, a threonine at position 120, a threonine at position 163, and a glutamine at position 581.

18. The cell or biologically pure culture of claim 15, wherein the amino acid sequence of the VHPO comprises an isoleucine at position 3, a tyrosine at position 69, a serine at position 75, an asparagine at position 81, a threonine at position 111, an alanine at position 138, an isoleucine at position 141, a lysine at position 147, a phenylalanine at position 288, a tyrosine at position 356, a leucine at position 575, a glutamine at position 581, a glutamine at position 594, and an alanine at position 598.

19. The cell or biologically pure culture of claim 15, wherein the amino acid sequence of the VHPO comprises an isoleucine at position 3, a tyrosine at position 69, a serine at position 75, an asparagine at position 81, an alanine at position 82, a threonine at position 111, a threonine at position 120, an alanine at position 138, an isoleucine at position 141, a lysine at position 147, a threonine at position 163, a lysine at position 215, an isoleucine at position 261, a phenylalanine at position 288, a tyrosine at position 356, a leucine at position 447, a lysine at position 538, an arginine at position 544, a leucine at position 575, a glutamine at position 581, a glutamine at position 594, an alanine at position 598, and an isoleucine at position 605.

20. (canceled)

21. An agricultural composition comprising the cell or a biologically pure culture of claim 14 or biomass from the cell or biologically pure culture, a culture supernatant from the cell or biologically pure culture, or a combination thereof, and,
optionally, an agriculturally acceptable carrier.

22. The agricultural composition of claim 21, further comprising a cereal, starch, vegetable waste, vitamin, mineral, trace element, emulsifier, aromatizing product, binder, colorant, odorant, thickening agent, or a combination thereof.

23. Animal feed comprising the agricultural composition of claim 22.

24-30. (canceled)
